(19) United States
(12) Patent Application Publication
(10) Pub. No.: US 2024/0363828 A1
(43) Pub. Date: Oct. 31, 2024

(54) PROCESS ARRANGEMENT AND METHOD FOR FABRICATING AN ELECTRODE FOR A BATTERY CELL

(71) Applicant: Volkswagen Aktiengesellschaft, Wolfsburg (DE)

(72) Inventor: Tobias ABEL, Braunschweig (DE)

(73) Assignee: Volkswagen Aktiengesellschaft, Wolfsburg (DE)

(21) Appl. No.: 18/646,718

(22) Filed: Apr. 25, 2024

(30) Foreign Application Priority Data
Apr. 26, 2023 (DE) 10 2023 203 853.6

(51) Int. Cl. H01M 4/04; B05C 9/12; B05C 9/14; G01N 27/04

(52) U.S. Cl. CPC H01M 4/0404; B05C 9/12; B05C 9/14; G01N 27/041; G01N 33/0078; H01M 4/0435

(57) ABSTRACT

A process arrangement for fabricating an electrode for a battery cell, in which a current collector film as a continuous web can be guided continuously through processing stations, namely while forming an electrode web that is disconnected and/or cut to size for the electrode at a final cutting station. The process arrangement has a measuring station with at least one measuring device in which the resistivity of an active material layer and/or the transfer resistance of the active material layer to the current collector film or a value correlating therewith can be measured.

This nonprovisional application claims priority under 35 U.S.C. § 119 (a) to German Patent Application No. 10 2023 203 853.6, which was filed in Germany on Apr. 26, 2023, and which is herein incorporated by reference.

### BACKGROUND OF THE INVENTION

### Field of the Invention

The invention relates to a process arrangement for fabricating an electrode for a battery cell and to a method for fabricating such an electrode.

### Description of the Background Art

The electrodes of a lithium-ion battery cell can be fabricated in large-scale production in a continuous process in which a current collector film as a continuous web is guided continuously through a coating station in which the current collector film is coated with active material on one or both sides. The electrode web thus formed then passes through a drying station, in which the active material coated on the current collector film is dried. Next follows a calendering station, in which the active material coated on the current collector film is compressed to a predefined layer thickness. The continuous process concludes at a cutting station, in which the electrode web is disconnected and/or cut to size for the electrode. The electrode thus produced is subsequently integrated into an electrode/separator stack, which can be incorporated into a battery cell.

The magnitude of the internal resistance of such a battery cell is important for the performance of the battery cell. The battery cell internal resistance is determined by factors that include the resistivity of the electrode active material and the transfer resistance of the active material layer to the current collector film. For measuring the resistivity of the active material layer and the transfer resistance, an analysis device of the HIOKO company is known (see “electrode resistance measurement system RM2610” brochure from the HIOKO company), with which the resistivity and the transfer resistance of already finished electrodes can be measured by sampling. If the analysis device detects an excessively high measured value for the resistivity or the transfer resistance, all electrodes from the entire coating batch of a production interval that have already been fabricated must be delivered to scrap material, and they therefore are no longer usable for further battery cell fabrication.

Known from DE 10 2014 006 870 A1 are a method and a device for measuring a coating thickness on a substrate. The method can specifically also be used for measuring the layer thickness of an active material layer on a current collector film for producing an electrode.

### SUMMARY OF THE INVENTION

It is therefore an object of the invention to provide a process arrangement and a method for fabricating an electrode for a battery cell in which quality testing of the electrode can be carried out with less waste material as compared with the prior art.

The invention is based on a process arrangement for fabricating an electrode for a battery cell. In the fabrication process, a current collector film as a continuous web is guided continuously through processing stations, namely while forming an electrode web. In a final cutting station, the electrode web is disconnected and/or cut to size for the electrode. A measuring station with a measuring device, in which the resistivity of an active material layer and/or the transfer resistance of the active material layer to the current collector film or a value correlating therewith is measured, is integrated in the process arrangement.

In contrast to the prior art, the measuring operation according to the invention does not take place only on the already-completed electrode. Instead, the measuring operation is carried out directly on the electrode web that has not yet been delivered to the cutting station, which is to say even before completion of the electrode. In this way, “in-line” quality testing of the resistivity or of the transfer resistance between film and coating is provided. If quality problems are identified in a section of electrode web in the measuring station, then this section of electrode web is delivered to scrap material while other electrode web sections (that tested as defect-free) of the same coating batch go through the normal process for fabricating the electrodes. In this way, the waste material in electrode production can be greatly reduced as compared with the prior art.

The process arrangement can have the following as a processing station: a coating station, in which a current collector film as a continuous web can be coated with active material on one or both sides; a drying station for drying the active material layer coated on the current collector film; and a calendering station, in which the active material coated on the current collector film is compressed to a predefined layer thickness. Indirectly or directly downstream of the calendering station is the cutting station, in which the electrode web is disconnected for the electrode.

The measuring station for measuring the resistivity and/or the transfer resistance can preferably be arranged directly before or after the calendering station. Depending on requirements, therefore, the resistivity as well as the transfer resistance can already be detected at an early point in time in the process, which is to say preferably even before the electrode web passes through the calendering station.

The measuring station integrated in the process arrangement can have an evaluation unit, in which a measured resistance value detected by the measuring device is compared with a nominal value stored in the evaluation unit. In the case of a significant deviation of the currently detected measured resistance value, a currently measured section of electrode web can be excluded from normal electrode manufacture and delivered to scrap material, while the other sections of electrode web with defect-free measured resistance value are used for further battery cell fabrication.

The measuring device can be realized in the manner of an analysis device from the HIOKI company (see “electrode resistance measurement system RM2610” brochure from the HIOKO company). In this case, the measuring device can have at least one microelectrode array whose electrodes are in nondestructive contact with the surface of the active material layer of the electrode web during the measurement. The microelectrode array can be subdivided into a voltage measuring array and a current measuring array. The voltage measurement and the current measurement can therefore be carried out separately from one another. With the aid of the microelectrode array, a section of electrode web can therefore be subjected to a predefined current flow at multiple points. The resultant potential distribution can be detected at multiple measurement points on the surface of the active material layer with the aid of the electrodes of the voltage measuring array. During the measuring operation, the current is not conducted solely through the current collector film, but also through the active material layer, so that both the transfer resistance and the resistivity can be measured.

A computing unit can be associated with the microelectrode array. Data from the measured current flow and the measured potential distribution, the layer thickness of the active material layer, and the resistivity of the current collector film are fed into the computing unit. In the computing unit, an FEM simulation based on these data takes place, by means of which the resistivity and the transfer resistance of the currently measured section of electrode web are determined.

The measuring station can have a roller arrangement with at least one measuring roller. The electrode web runs over the measuring roller. The measuring device can be arranged directly on the outer circumference of the measuring roller in this case, by which means “in-line” quality testing is made possible that is simple in process engineering terms.

With regard to quality testing with high process reliability, the measuring device can be arranged not directly on a rigid body of the measuring roller, but instead be arranged on the base material of the measuring roller with the interposition of an elastically resilient cushioning material. The cushioning material acts in the manner of an overload protection spring so that the measuring device always presses against the active material layer of the continuous web with a predefined contact pressure that is determined by the spring constant of the elastically resilient cushioning material. The measuring operation can be performed reliably in this case regardless of tolerance deviations in the layer thickness of the active material layer.

The outer circumference of the measuring roller can be covered with a wear-resistant coating outside the measuring device. In this way, roller material wear can be avoided that would lead to contamination of the active material layer.

To increase measurement accuracy, the roller arrangement can have two measuring rollers over which the electrode web runs with both of its sides. In this case, one side of the electrode web can be brought into measurement contact with the first measuring roller, while the other side of the electrode web can be brought into measurement contact with the second measuring roller.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIGS. 1 to 5 show different views illustrating a measuring station according to the invention of the process arrangement.

### DETAILED DESCRIPTION

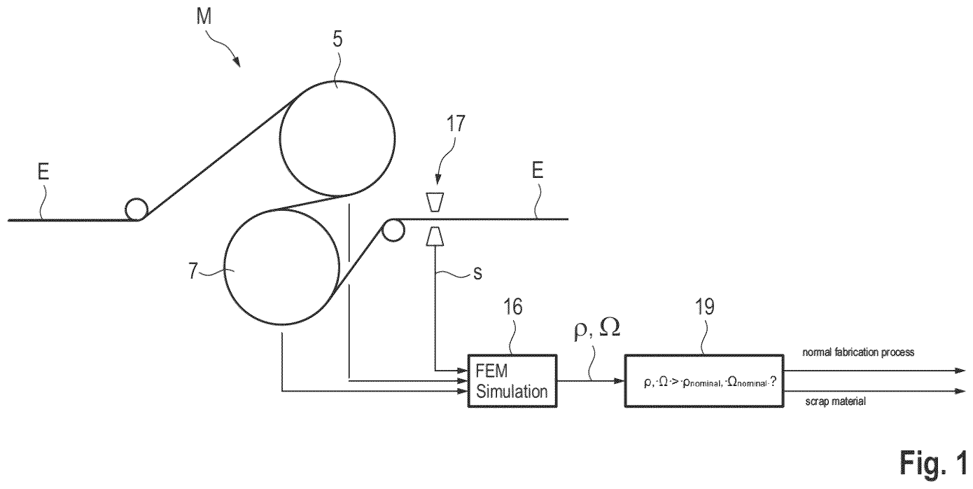

Shown in FIG. 1 is a measuring station M for quality testing that is part of a process arrangement, in which electrodes for a battery cell are fabricated in a continuous process. In addition to the measuring station M, the process arrangement has other processing stations that are not shown, namely a coating station, a drying station, a calendering station, and a cutting station. In the coating station, a current collector film 1 as a continuous web is coated on one or both sides with an active material layer 3, namely while forming an electrode web E. In the further course of the process, the electrode web E is guided through the drying station, in which the active material layer 3 coated on the current collector film 1 is dried. After that, the electrode web E is guided through the calendering station, in which the active material layer 3 coated on the current collector film 1 is compressed to a predefined layer thickness. Next follows the cutting station, in which the electrode web E is disconnected and also cut to size for the electrode.

The resistivity p of the active material layer 3 as well as the transfer resistance Q of the active material layer 3 to the current collector film 1 can be measured with the aid of a measuring device 4 of the measuring station M. The essence of the invention includes that the measuring station M is integrated in the process arrangement for continuous electrode fabrication. As a result, the measuring operation can be carried out directly on the electrode web E “in line,” or in other words during the continuous fabrication process, which is to say as early as a point in time in the process before completion of the electrode.

As is further evident from FIG. 1, the measuring station M has a roller arrangement composed of two measuring rollers 5, 7 over which the electrode web E runs with both of its sides. This means that, according to FIG. 1, one side of the electrode web E can be brought into measurement contact with the first measuring roller 5, while the other side of the electrode web E can be brought into measurement contact with the second measuring roller 7. The measuring rollers 5, 7 have the measuring devices 4, which are positioned on the outer circumference of each of the measuring rollers 5, 7 in a circumferentially distributed manner. Current-carrying contacts of the measuring devices 4 can be integrated in the relevant measuring roller 5, 7 in a wired manner or by means of a battery solution, for example. Moreover, a precision thickness measurement 17, by means of which a local thickness of the electrode web E is detected, is downstream of the two measuring rollers 5, 7 in FIG. 1.

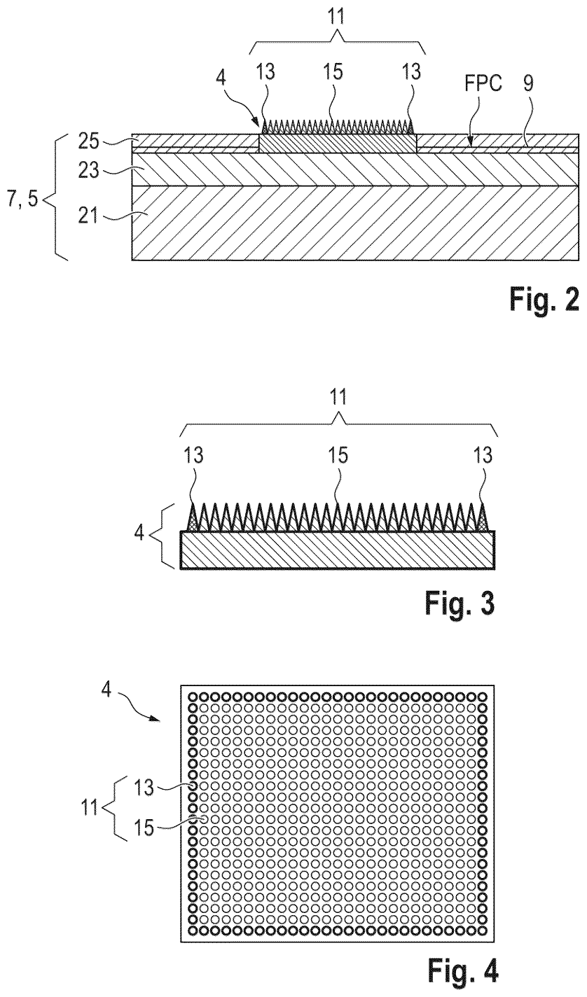

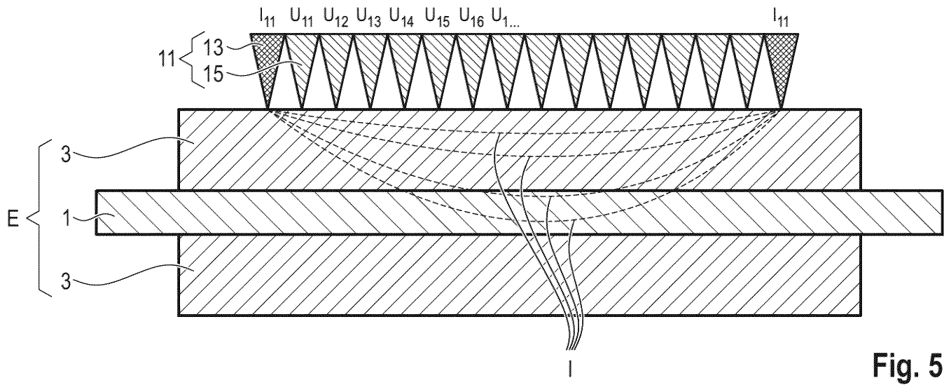

One measuring device 4 of the measuring devices 4 is shown in detail in FIGS. 2 to 5. This device is implemented as a chip with electrode array 11, which is shown schematically in FIGS. 2 to 4 to the extent necessary for understanding the invention. The overall width of the chip 4 is dimensioned in the order of magnitude of the coating thickness. Moreover, the chip 4 according to FIG. 2 is part of a flexible printed circuit design (flexible printed circuit) FPC, which is formed of a thin, flexible plastic substrate 9 onto which are applied electrical conductive traces as well as the chip 4. The chip 4 has a microelectrode array 11, which is subdivided into a current measuring array 13 and a voltage measuring array 15. The current measuring array 13 formed of outer current measurement electrodes on the edges, while the voltage measuring array 15 formed of inner voltage measurement electrodes that are surrounded by the outer current measurement electrodes. The current measurement and the voltage measurement therefore take place independently of one another by means of the current measuring array 13 and the voltage measuring array 15. During a measuring operation (FIG. 5), the electrodes of the microelectrode array 11 are in nondestructive contact with the surface of the active material layer 3 of the electrode web E. A section of electrode web that is currently to be measured is subjected to a predefined current flow I at multiple points with the aid of the microelectrode array 11. The resultant potential distribution is detected at multiple measurement points on the surface of the active material layer 3 by the electrodes of the voltage measuring array 15.

The electrodes of the outer current measuring array 13 can be used as either a current source or sink during the measuring operation, while the electrodes of the inner voltage measuring array 15 can be used for a voltage measurement. In this design, each of the electrodes can have a separately controllable or readable channel (this can also be coded). The signal connection of the electrodes of the chip 4 to the evaluation unit 19 can be implemented through wireless (5G, Wifi) or wired inductive contacts after digital coding of the signals.

Associated with the measuring device 4 in FIG. 1 is a computing unit 16, into which the data detected by the current measuring array 13 and the voltage measuring array 15 and the data detected by a thickness measurement 17 of the electrode web E (which is to say the measured electrode web thickness s) can be read. Based on these data and the resistivity of the current collector film 1, the computing unit 16 determines the resistivity p of the active material layer 3 and the transfer resistance Q of the active material layer 3 to the current collector film 1 of the currently measured section of electrode web. The computing unit 16 operates by means of a local FEM simulation. For this reason, a simulation of the active material layers 3 located under the chip 4 takes place in the computing unit 16 with the aid of a simulation model and the layer thickness measurement.

The determined resistance values p, Q are delivered to an evaluation unit 19, which compares the resistance values p, Q with corresponding nominal values ρₙₒₘᵢₙₐₗ, Ωₙₒₘᵢₙₐₗ. In the case of a significant deviation of the currently detected measured resistance values, the currently measured section of electrode web is not used for further electrode fabrication, but instead is delivered to scrap material. If the relevant measured resistance values p, Q are in the region of the nominal values ρₙₒₘᵢₙₐₗ, Ωₙₒₘᵢₙₐₗ without relatively large deviation, the currently measured section of electrode web is used for further battery cell fabrication.

In FIG. 5, a current flow I between current measuring electrodes of the current measuring array 13 is indicated with dashed lines. Accordingly, the current I does not flow solely through the current collector film 1, but also through the active material layer 3, so that both the resistivity ρ of the active material layer 3 and the transfer resistance Ω of the active material layer 3 to the current collector film 1 can be measured with great accuracy.

The material construction of the measuring rollers 5, 7 is shown roughly schematically in FIG. 2. Accordingly, the relevant measuring roller 5, 7 has a rigid base material 21. The flexible printed circuit design FPC is not arranged directly on the rigid base material 21 of the measuring roller 5, but instead with the interposition of an elastically resilient cushioning material 23. The cushioning material 23 acts in the manner of an overload protection spring, by means of which the measuring device 4 (which is to say the chip) always presses against the active material layer 3 of the continuous web E with a predefined contact pressure, namely independently of, e.g., tolerance deviations in the layer thickness s of the electrode web E. Moreover, the measuring roller 5 is covered on the outer circumference with a wear-resistant coating 25 (made of PTFE, for example) outside the measuring device 4, by which means operationally caused roller material wear is avoided that could lead to contamination of the active material layer 3.

The chip 4 can also be applied directly to the measuring roller 5, 7 or embedded in a firm layer, for example. Furthermore, the roller construction can also be implemented without a wear-resistant layer 25. Moreover, instead of PTFE, the wear-resistant layer 25 can also be made of metals, such as chrome or nickel, or of ceramic coatings. Moreover, another example is a chip with individual spring-loaded contacts. This can be ensured by any desired type of spring. What is important is that there is no electrical contact between the different electrodes during operation.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A process arrangement for fabricating an electrode for a battery cell, in which a current collector film as a continuous web is guided continuously through processing stations while forming an electrode web that is cut to length and/or cut to size for the electrode at a final cutting station, the process arrangement comprising:
a measuring station with at least one measuring device in which the resistivity of an active material layer and/or a transfer resistance of the active material layer to the current collector film or a value correlating therewith is measured,
wherein a measuring operation is carried out directly on the electrode web before a completion of the electrode.

2. The process arrangement according to claim 1, wherein the process arrangement comprises, as processing stations:
a coating station, in which the current collector film is coated on one or both sides with the active material layer;
a drying station for drying the active material layer coated on the current collector film; and
a calendering station, in which the active material layer coated on the current collector film is compressed to a predefined layer thickness,
wherein the cutting station is downstream of the calendering station in process engineering terms.

3. The process arrangement according to claim 1, wherein the measuring station is directly upstream and/or downstream of the calendering station in process engineering terms, so that the resistivity or the transfer resistance is measured before and/or after the calendering procedure.

4. The process arrangement according to claim 1, wherein the measuring station has an evaluation unit that compares a measured resistance value detected by the measuring device with a nominal value, and wherein, in a case of a significant deviation of the currently detected measured resistance value, the currently measured section of electrode web is not used for electrode fabrication, but instead is delivered to scrap material.

5. The process arrangement according to claim 1, wherein the measuring device has at least one microelectrode array whose electrodes are in nondestructive contact with the surface of the active material layer during the measurement.

6. The process arrangement according to claim 5, wherein a section of electrode web is subjected to a predefined current flow at multiple points via the microelectrode array, and wherein the resultant potential distribution is detected at multiple measurement points on the surface of the active material layer, and/or wherein the microelectrode array is subdivided into a current measuring array, the electrodes of which act as a current source and current sink to create the current flow, and a voltage measuring array, the electrodes of which measure the potential distribution resulting from the current flow, and/or wherein, associated with the microelectrode array is a computing unit that computes the resistivity of the active material layer as well as the transfer resistance on the basis of the current flow, the detected potential distribution, the layer thickness of the active material layer, and the resistivity of the current collector film, namely via an FEM simulation.

7. The process arrangement according to claim 1, wherein the measuring station has a roller arrangement with at least one measuring roller over which the electrode web runs, and wherein the measuring device is arranged on the outer circumference of the measuring roller, or wherein the measuring station has a linearly displaceable carriage that is motion-coupled to the electrode web, via which the measuring device is be brought into measurement contact with the electrode web over a linear measurement path.

8. The process arrangement according to claim 7, wherein the measuring device is not arranged directly on a rigid base material of the measuring roller, but instead is arranged on the base material of the measuring roller with the interposition of an elastically resilient cushioning material so that the measuring device presses against the active material layer of the electrode web with a predefined contact pressure independently of tolerance deviations in the layer thickness of the active material layer, and wherein the outer circumference of the measuring roller is covered with a wear-resistant coating in a region outside the measuring device in order to avoid contamination of the active material layer owing to roller material wear, and/or wherein the roller arrangement has two measuring rollers over which the electrode web runs with both of its sides so that one side of the electrode web is brought into measurement contact with the first measuring roller and the other side of the electrode web is brought into measurement contact with the second measuring roller.

9. A method for fabricating an electrode in a process arrangement according to claim 1.
